(19) United States
(12) Patent Application Publication
(10) Pub. No.: US 2024/0087112 A1
(43) Pub. Date: Mar. 14, 2024

(54) REPRESENTING A BIOLOGICAL IMAGE AS A GRID DATA-SET

(71) Applicant: CBMED GMBH CENTER FOR BIOMARKER RESEARCH IN MEDICINE, Grax (AT)

(72) Inventor: Laurin Herbstofer, Grosswilfersdorf (AT)

(21) Appl. No.: 18/263,506

(22) PCT Filed: Feb. 5, 2021
(86) PCT No.: PCT/EP2021/052792

§ 371 Date: Jul. 28, 2023

(51) Int. Cl. G06T 7/00; G06T 7/11; G06V 10/44; G16H 50/20

(52) U.S. Cl. CPC G06T 7/0012; G06T 7/11; G06V 10/44; G16H 50/20; G06T 2207/10056; G06T 2207/30024; G06V 2201/07

(57) ABSTRACT

The present invention relates to a method for processing an image. Moreover, a grid data set, an output image, different uses of a grid data set and an output image, a device, a computer program, a computer-readable medium, a neural network, a method for diagnosing a disease, a method for prognosing the course of a disease, a method for determining whether a patient suffering from a disease will respond to a therapeutic treatment and a method for predicting relapse of a disease are specified.

A method for processing an image is specified. Moreover, a grid data set, an output image, different uses of a grid data set and an output image, a device, a computer program, a computer-readable medium, a neural network, a method for diagnosing a disease, a method for prognosing the course of a disease, a method for determining whether a patient suffering from a disease will respond to a therapeutic treatment and a method for predicting relapse of a disease are specified.

### BACKGROUND OF THE INVENTION

Images, e.g. images of biological tissues, can be of large size. A micrograph of a biological tissue section at 20× magnification will show the individual biological cells that constitute the tissue. At a resolution of 0.5 μm, a single biological cell with a diameter of 10 μm is captured by several hundred pixels in the image. For many biological and clinical questions, this subset information is less relevant than the phenotype of the cells and their relative location in the tissue. Furthermore, a whole-slide-image (WSI) of a single tissue slide with a diameter of 1 cm can be several Gigabytes large, while only including about a million biological cells. This makes large-scale data sharing within the research community challenging.

Neural networks, like convolutional neural networks (CNNs), may be used with those images including, e.g., hematoxylin-eosin (HE)-stained tissue slides as well as (multiplex) immunohistochemistry (IHC) stainings. The neural networks may be trained to make predictions, diagnoses etc. when using those images as an input. However, training neural networks with such images is time-consuming and needs large computational resources. Moreover, such neural networks may be difficult to interpret for humans.

It should be noted that the statements above should not be construed as being admitted prior art. They are only made to illustrate the background of the presently disclosed concepts and may not have been made available to the public yet.

### DETAILED DESCRIPTION OF THE INVENTION

One object to be achieved is, inter alia, to provide an improved method for processing such an image, in the following also referred to as initial image. The method preferably allows or is configured to reduce the size of the initial image so that an accordingly obtained output image or output data set may be used to efficiently train a neural network, said neural network preferably also allowing a better interpretation by human.

Further objects to be achieved are to provide an improved output data set, also referred to as grid data set in the following, e.g. produced with such a method, an improved output image, e.g. produced with such a method, different uses of a grid data set or an output image, a device for carrying out the method, a computer program for carrying out the method, a computer-readable medium for carrying out the method, a neural network trained with such a grid data set and/or such an output image and methods for diagnosing a disease, predicting the course of the disease, determining whether a patient suffering from a disease will respond to a therapeutic treatment and for predicting relapse of a disease in a patient by using such a grid data set and/or such an output image.

Firstly, the method for processing an image is specified.

According to at least one embodiment, the method comprises a step in which an n-dimensional grid is assigned to a distribution of objects in the n-dimensional space. Here and in the following, when talking about n dimensions, n spatial dimensions are meant in particular. For example, n may be equal to 1 or 2 or 3.

According to at least one embodiment, n is greater or equal 2.

According to at least one embodiment, the distribution of the objects is derivable or is derived from an object data set. The object data set is, e.g., indicative for an arrangement of the objects in an n-dimensional initial image. For example, the object data set is indicative for spatial positions, e.g. the center-of-mass positions, of the objects in the initial image. For example, the object data set comprises information of or is indicative for at least 25 or at least 100 or at least 1000 or at least 10,000 objects and their spatial distribution. The objects may all be of the same type or genus, e.g. they may all be biological cells.

“Spatially assigning” may mean that the distribution of the objects and the grid are virtually overlapped or aligned or fixed with each other. The grid is preferably chosen such that at least 50 or at least 75% or at least 90% or at least 99% of the spatial distribution of the objects can be assigned to or overlapped with grid. E.g. the volume of the grid in the n-dimensional space is at least 50% or at least 75% or at least 90% or at least 99% of the volume occupied by the spatial distribution of the objects.

According to at least one embodiment, the grid comprises grid cells and each grid cell is associated with a position in the grid. Preferably, each grid cell is assigned a cell position on a one-to-one basis. Each grid cell may be assigned a grid node of the grid on a one-to-one basis. The cell positions of the grid cells may be the centers of the grid cells or may be the positions of the assigned grid nodes.

For example, the grid comprises at least 25 or at least 100 or at least 1000 or at least 10,000 grid cells. For example, the number of grid cells is at least 10% or at least 50% or at least 90% of the number of objects in the object data set. Additionally or alternatively, the number of grid cells is at most 200% or at most 150% or at most 110% of the number of objects in the object data set.

Here and in the following, assigning first elements to second elements on a one-to-one basis means that each first element is assigned to at most one second element and at most one first element is assigned to each second element.

The grid is, e.g., a mathematical and/or virtual grid composed of bins wherein each bin constitutes a grid cell. The grid is preferably a grid in the Euclidean space.

According to at least one embodiment, the method comprises a step, in which objects are assigned to grid cells depending on the relative spatial arrangement between the objects and the grid cells. This step is preferably performed after the grid has been spatially assigned to the distribution of the objects. The objects may be assigned to grid cells depending on the distance between the objects and the grid cells or depending on the spatial overlap between the objects and the grid cells. In this step, one or more grid cells may not be assigned objects. Those grid cells may remain empty. Likewise, one or more objects may not be assigned to grid cells. However, preferably at least 50% or at least 90% of objects are assigned to grid cells in this step.

In at least one embodiment, the method for processing an image comprises a step in which an n-dimensional grid is spatially assigned to a distribution of objects in the n-dimensional space, wherein n is greater or equal 2. The distribution of objects is derivable from an object data set and the object data set is indicative for an arrangement of the objects in an n-dimensional initial image. The grid comprises grid cells and each grid cell is associated with a cell position in the grid. In a further step, objects are assigned to grid cells depending on the relative spatial arrangement between the objects and the grid cells.

By assigning objects from an initial image to grid cells of a grid, data compression can be achieved. For example, the initial image has a higher resolution than the grid. Only the relevant information from the initial image, e.g. special feature of the objects and/or the positions of the objects, are extracted from the initial image and are then assigned to grid cells so that an output image can be generated with at least some grid cells each representing one complete object of the initial image, wherein the output image has a resolution defined by the density of grid cells in the grid. Important information of the initial image, e.g. the position of the objects in the initial image and eventually further features from the objects (object features) are maintained or approximately maintained. The output image and/or the corresponding data (also referred to as grid data set) may be used to more efficiently train a neural network. The compression obtained with the method may lead to much smaller neural networks, e.g. CNNs, (i.e. fewer trainable parameters), which is beneficial for training (shorter training time, less powerful hardware is required).

According to at least one embodiment, the method is a computer-implemented method, i.e. it is executed or performed by a computer.

According to at least one embodiment, the object data set is indicative for the positions of the objects in the n-dimensional initial image. Here and in the following, when data are indicative for a certain feature, this particularly means that information about this feature are stored in the data and/or can be extracted from the data. For example, the object data set comprises, at least for some or all objects, the information about the position of the respective object. The position of the object may be the center-of-mass position of the object or a position of a certain feature of the object, e.g. the position of a nucleus of the object.

According to at least one embodiment, the objects are assigned to the grid cells depending on the positions of the objects relative to the cell positions. For example, the objects are assigned to the grid cells depending on the distances between the positions of the objects and the cell positions.

The object data set itself may be obtained from an initial image by identifying objects in the initial image and, e.g., determining the positions of the objects. Identifying of the objects may be done with commercial software. The object data set may already be reduced in size compared to the initial image as it may not comprise information about every pixel from the initial image but only information about the objects in the initial image, e.g. the positions of the objects and eventually one or more further object features.

According to at least one embodiment, objects are assigned to grid cells on a one-to-one basis.

According to at least one embodiment, for assigning the objects to grid cells, a first assignment procedure is executed in which objects are assigned to the respective closest grid cell. For this purpose, the distances between the positions of the objects and the grid cell positions may be used in order to determine, for each object, which grid cell is the closest. Alternatively, in the first assignment procedure, objects may be assigned to the grid cells depending on the spatial overlap between the objects and the grid cells. E.g., an object is assigned to that grid cell for which the spatial overlap is largest.

According to at least one embodiment, if, in the first assignment procedure, two or more objects are to be assigned to the same grid cell, these objects constitute conflicting objects. The respective grid cell constitutes a conflict grid cell. In this case, a conflict resolution procedure may be executed. The conflict resolution procedure may be executed in order to assign the conflicting objects to different grid cells, preferably such that the conflicting objects are assigned to grid cells on a one-to-one basis, and/or it may be decided to not assign at least one of the conflicting objects to any grid cell. When assigning the conflicting objects to different grid cells, one of these grid cells is preferably the conflict grid cell.

According to at least one embodiment, at least some objects are assigned to grid cells using the Hungarian algorithm, also known as Kuhn-Munkres algorithm or Munkres assignment algorithm. The Hungarian algorithm is, e.g., disclosed and explained in the papers by Kuhn, H. W. (1955), The Hungarian method for the assignment problem, Naval Research Logistics, 2: 83-97. https://doi.org/10.1002/nay.3800020109 and Munkres, J. (1957), Algorithms for the Assignment and Transportation Problems. Journal of the Society for Industrial and Applied Mathematics, 5(1), 32-38, Retrieved Jan. 20, 2021, from http://www.jstor.org/stable/2098689. The disclosure content of these papers is hereby incorporated by reference.

According to at least one embodiment, an assignment matrix C, also known as cost matrix C, is used for the Hungarian algorithm, wherein the values of the elements cij of the matrix C dependent on the distance between the i-th object to the j-th grid cell. The distance between the position of the object and the cell position may be used as the distance between the object and the grid cell.

According to at least one embodiment, the values of the elements cij are proportional to or a function of the distance between the i-th object to the j-th grid cell. For example, the values of the elements cij are proportional to or a function of the squared distance between the i-th object to the j-th grid cell.

According to at least one embodiment, the distance between the i-th object and the j-th grid cell is the Euclidean distance or the Manhattan distance. The Manhattan distance is also known as the L1 distance.

If the Hungarian algorithm is used for assigning k objects to m grid cells (wherein m and k are integers) and if k is not equal to m, the assignment matrix may be supplemented with default elements cij(def) to make the matrix square and to take into account the excess of objects or grid cells. The default values of these default elements cij(def) may be chosen to be 0. This is equivalent to add virtual objects or virtual grid cells, respectively, in order to have the same amount of grid cells and objects. When k is larger than m, those objects which are assigned to virtual grid cells when solving the Hungarian algorithm may be deleted. When m is larger than k, those grid cells which are assign a virtual object, may finally remain empty.

According to at least one embodiment, the conflict resolution procedure comprises the execution of the Hungarian algorithm in order to assign the conflicting objects, and preferably also objects in the neighborhood of the conflicting objects, to a cell-set comprising the conflict grid cell and one or more selected grid cells in the neighborhood of the conflict grid cell. The neighborhood of the conflict grid cell may be chosen such that the selected grid cells of the cell-set are symmetrically distributed around the conflict grid cell, e.g. with the conflict grid cell forming a center of point symmetry of the cell-set. For example, the cell-set comprises all grid cells within a certain contour or a certain radius around the conflict grid cell or within a square or cube, the center of the square/cube being in the center of the conflict grid cell.

According to at least one embodiment, when executing the conflict resolution procedure, an object-set is determined comprising objects which would have to be assigned to the grid cells of the cell-set when executing the first assignment procedure. For example, the object-set comprises all objects within the contour or the radius or the square/cube around the conflict grid cell.

According to at least one embodiment, after determining the object-set, only objects of the object-set are assigned to the grid cells of the cell-set by using the Hungarian algorithm. In other words, the Hungarian algorithm is only used locally for resolving the conflict.

According to at least one embodiment, if the number k of objects in the object-set is larger than the number m of grid cells in the cell-set, the neighborhood of the conflict grid cell is increased in order to increase the amount of selected grid cells until m is greater or equal k or until m reaches a predetermined maximum value m_max. For example, in a first step, the cell set comprises 9 grid cells, in a second step, the cell-set comprises 25 grid cells, in a third step, the cell-set comprises 49 grid cells and so on. If, after m having reached the value m_max, k is still larger than m, k-m objects may be deleted, i.e. are not assigned to grid cells. The information from these deleted objects may be lost.

According to at least one embodiment, m_max is smaller than the total number of grid cells in the grid, e.g. m_max is at most 1% or at most 10% of the total number of grid cells.

According to at least one embodiment, m_max is at most 100 or at most 81 or at most 64 or at most 49. Too large values of m or m_max, respectively, increase computation time for solving the Hungarian algorithm. Therefore, it may be advantageous to have m or m_max to not be too large.

After a conflict has been resolved for one conflict grid cell by using the conflict resolution procedure, a next conflict grid cells may be considered. For the next conflict grid cell, also the conflict resolution procedure may be applied to resolve the conflict. This may be done until all conflicts are resolved. The order in which the conflict grid cells are considered may be arbitrary.

Objects which have been assigned to grid cells in a previous conflict resolution procedure may be blocked (not used) for the following conflict resolution procedures. Alternatively, these objects may not be blocked and may again be used for the following conflict resolution procedures.

Instead or additionally of executing the Hungarian algorithm during the conflict resolution procedure, a priority shift may be executed in order to resolve the conflict. In this priority shift, the conflicting object closest to the conflict grid cell or having the largest overlap with the conflict grid cell is assigned to the conflict grid cell. One or more of the remaining conflicting objects are then assigned to the next free grid cell, i.e. to a grid cell to which no object has been assigned so far. For assigning the one or more of the remaining conflicting objects, only empty grid cells in a certain neighborhood, e.g. within a predetermined radius around the conflict grid cell, may be considered

According to at least one embodiment, the grid is a regular grid. The grid may be a square grid or a rectangular grid or a hexagonal grid. All grid cells of the grid may have the same shape and/or the same size and/or the same volume. The grid cells may be squares or rectangles or hexagons.

According to at least one embodiment, the initial image has a first spatial resolution defined by the pixel size of the initial image. For example, the pixel size of the initial image is at most 1 μm or at most 0.5 μm or at most 0.25 μm.

According to at least one embodiment, the grid has a second spatial resolution defined by the grid cell size. The grid cell size may be equal to a lattice constant of the grid or may be the maximal extension of a grid cell in one spatial direction. The grid cell size may be at least 1 μm or at least μm or at least 10 μm. Additionally or alternatively, the grid cell size may be at most 20 μm.

According to at least one embodiment, the pixel size is smaller than the grid cell size. For example, the grid cell size is at least 10 times or at least 25 times or at least 100 times or at least 1000 times or at least 10,000 times the pixel size.

According to at least one embodiment, the grid has a lattice constant d. The lattice constant d may be equal to the length or the size of the grid cells.

According to at least one embodiment, the lattice constant d is chosen to depend on rho or to be a function of rho, respectively. For example, the lattice constant d is proportional to rho\hat{ }(−1/n). Rho is the density of objects in the initial image. As described before, n stands for the number of spatial dimensions of the initial image and of the distribution of objects.

For example, d may be chosen depending on the expected fraction r of objects which have to be deleted when using the conflict resolution procedure for a cell-set with w\hat{ }2 grid cells (w\hat{ }2 is an integer). The expected fraction r is calculated as follows

$$r ( w , d ) = 1 - \frac{{w}^{2}}{λ ( w d )} - \frac{{w}^{2}}{λ ( w d )} {\sum }_{k = 0}^{{w}^{2}} P o i s ( λ ( wd ) , k ) \cdot ( \frac{k}{{w}^{2}} - 1 ) ,$$

$$wherein$$

$$λ ( wd ) = rho \cdot {w}^{2} \cdot {d}^{2} and Pois ( λ , k ) = \frac{{λ}^{k}}{k !} {e}^{- λ} .$$

This formula holds for n is equal to 2 and has to be adapted for other values of n.

For example, a maximum value of r can be predetermined or provided by a user. For example, r is set to at most 1% or at most 0.5% or at most 0.1%. Using this value of r, the above-mentioned formula allows to calculate a lattice constant d which may then be used as the maximum lattice constant d for the grid.

Alternatively, a compression ratio R may be predetermined or provided by the user and the lattice constant d may be determined based on the compression ratio R. For example, the lattice constant d then calculates as follows:

d=s·sqrt(R),

wherein s is the pixel size of the initial image.

Additionally or alternatively, d may be chosen to be at most D/sqrt(2) with D being the average equivalent spherical diameter of the objects. For example, in order to determine D, it is averaged over all objects of the initial image.

According to at least one embodiment, the lattice constant d is between 2 μm inclusive and 10 μm inclusive, e.g. between 2 μm inclusive and 6 μm inclusive.

According to at least one embodiment, the object data set is indicative for one or more object features being characteristic for the objects. The object features of an object are preferably different from the position of the object.

According to at least one embodiment, the method comprises a step in which a grid data set is produced. The grid data set may be produced by assigning one or more object features of the objects to the grid cells to which the objects are assigned. E.g., to each grid cell to which an object is assigned, one or more object features of the respective object are assigned.

According to at least one embodiment, the grid data set is indicative for the cell positions of all grid cells of the grid and also indicative for which object features are assigned to which grid cells. In other words, the grid data set comprises information about the cell positions of all grid cells and information about to which grid cell which object features are assigned.

According to at least one embodiment, the method comprises a step in which an n-dimensional output image is produced depending on the grid data set. The output image may be indicative for the initial image and shows one or more object features at the respective cell positions. Particularly, the output image is a n-dimensional visual representation of the grid data set. The output image shows objects or object features, respectively, in the grid cells or at the cell positions, respectively. The resolution of the output image is defined by the lattice constant of the grid and is, preferably, lower than the resolution of the initial image.

According to at least one embodiment, the method comprises a step in which several grid data sets are combined. Each grid data set is preferably obtained from a different object data set, wherein each object data set, in turn, is preferably indicative for a different initial image.

According to at least one embodiment, the method comprises a step in which an n+L-dimensional output image is produced depending on the combined grid data sets. Preferably, L is a number greater or equal 1. The n+L-dimensional output image may be indicative for the initial images and shows one or more object features of the objects at the respective cell positions.

The n+L dimensions particularly stand for n+L spatial dimensions. Preferably, n+L is equal to 3.

According to at least one embodiment, the different initial images are images from different sections or slices of the same sample, e.g. from a tissue sample of a patient. The lattice constant of the grid(s) may be chosen to be equal or approximately equal to the average thickness of the slices, e.g. with a deviation of at most 10% from the average thickness.

According to at least one embodiment, the method comprises a step in which a neutral network, e.g. a convolutional neural network, CNN for short, is trained with the grid data set and/or the output image (the n-dimensional and/or the n+L-dimensional output image). The neural network may be trained to produce an output depending on an input. The input may have the same structure as the grid data set and/or the output image or may be another grid data set and/or another output image produced with the method specified above.

The neural network may be trained with a plurality of grid data sets and/or output images, e.g. with at least 10 or at least 100 or at least 1000 grid data sets and/or output images. Preferably, each grid data set and/or output image is obtained from a different initial image or a different object data set being indicative for an initial image, respectively. Each of the grid data sets and/or the output images may be produced with the method specified above.

Additionally or alternatively, the neural network may be trained with one or more synthetic grid data sets and/or synthetic output images.

Training a neural network with the grid data set and/or the output image may be much more efficient than training a neural network with the initial image since the output image comprises less information than the initial image, preferably all relevant information the neural network needs for producing a desired output.

According to at least one embodiment, the grid data set and/or the output image is transformed by one or more of the following augmentation steps in order to increase the sets for training the neural network:
translation (in one or more directions, e.g. by max 20 or 30 grid cells),
reflection (horizontal and/or vertical reflection),
discrete rotation (e.g. by 90° and/or 180° and/or 270°),
deletion of objects in a certain area (e.g. of 25×25 grid cells) and at a random location,
local shuffling of objects in a certain area (e.g. of 3×3 grid cells and for 50 random locations),
changing of object features (e.g. change of brightness individually for each color channel and/or same change of brightness for all color channels),
deletion of object features in grid cells,
discrete shearing in x or y direction (e.g. by max 20 grid cells),
discrete shear rotation (e.g. by max 180°).

According to at least one embodiment, the neural network is trained to produce an output which is one or more of: diagnosing a disease in a patient, prognosing the course of a disease in a patient, predicting whether patients suffering from a disease will respond to a therapeutic treatment of said disease, predicting relapse of a disease in a patient.

According to at least one embodiment, the method further comprises a step in which one or more network features are extracted from the trained neural network. The network features may be indicative for why the neural network delivers a specific output for a specific input. The network features may be human readable features, e.g. biomarkers. For example, a network feature may be the number of cytotoxic T cells within a certain radius, e.g. of 10 μm, around a tumor cell. This number may give a hint on the relapse risk.

According to at least one embodiment, the method comprises a step in which the grid data set and/or the output image (the n-dimensional and/or the n+L dimensional output image) is used as an input for a neural network, e.g. of the trained neural network as specified above. The neural network may be trained to produce an output based on the input. The output may be one or more of the above-mentioned outputs of the trained neural network.

According to at least one embodiment, the objects (20) are biological cells and the initial image (2) is an image of a biological sample (4). The biological sample may be obtained from/isolated from/of a patient or reference subject. Thus, the initial image (2) may be an image of a biological sample (4) obtained from/isolated from/of a patient or reference subject.

The term “patient” may mean any individual whose image of a biological sample needs to be analyzed. In addition, the term “patient” may mean any individual for whom it is desired to know whether she or he suffers from a disease or disorder. In particular, the term “patient” may refer to an individual suspected to be affected by a disease or disorder. The patient may be diagnosed to be affected by the disease or disorder, i.e. diseased, or may be diagnosed to be not affected by the disease or disorder, i.e. healthy. The patient may further be prognosed to develop a disease or disorder. The term “patient” may also refer to an individual which is affected by a disease or disorder, i.e. diseased. The patient may be retested for the disease or disorder and may be diagnosed to be still affected by the disease or disorder, i.e. diseased, or not affected by the disease or disorder anymore, i.e. healthy, for example after therapeutic intervention. The patient may be a human or an animal. Human patients are particularly preferred.

The term “reference subject” may mean any individual whose image of a biological sample has/had been analyzed for control/comparative purposes. In addition, the term “reference subject” may mean any individual known be affected by a disease or disorder (positive control), i.e. diseased, or known to be not affected by a disease or disorder (negative control), i.e. healthy. The reference subject may be a human or an animal. Human reference subjects are particularly preferred.

According to at least one embodiment, the one or more object features comprise: size of the biological cell, shape of the biological cell, marker distribution properties in the biological cell, average marker intensity in the biological cell, size of the nuclei of the biological cell, and/or shape of the nuclei of the biological cell.

All cells express characteristic markers (proteins, lipids, glycosylation, etc.) or have specific morphological/structural characteristics that can be used to help distinguish unique cell types. Cell markers can be expressed both extracellularly on the cells surface or as an intracellular molecule.

Any cell, e.g. immune cell, stem cell, or central nervous system cell, can be characterized/identified by its specific surface and intracellular cell markers.

The term “marker” may refer to any marker stainable/detectable on/within a biological cell, e.g. any marker stainable/detectable with antibody-based methods.

The terms “average marker intensity” or “average marker expression” may mean the marker intensity or marker expression averaged over the whole/entire cell and that for each marker. Since some markers can stain targets that are found on the membrane, in the cytosol and/or in the nucleus, the terms “average marker intensity” or “average marker expression” may also mean the marker intensity or maker expression averaged over the cell membrane, cytosol, and/or nucleus”.

The term “marker distribution properties in the biological cell” may mean only the “properties of the distribution of the marker intensity” within a cell, i.e. high variance versus more uniform distribution.

Said markers may be cell surface markers or intracellular markers. Said markers are preferably tumor cell markers and/or immune cell markers. Said tumor cell markers may be HER2, Programmed death ligand 1 (PD-L1), or Programmed death protein 1 (PD-1). Said immune cell markers may be CD3, CD4, CD8, CD45, CD45RO, or forkhead box P3 (FoxP3). However, also other cell surface markers or intracellular markers may be used.

According to at least one embodiment, the initial image (2) is an image of a biological cell.

In one preferred embodiment, the above-mentioned biological cells are human, animal or plant cells.

In one preferred embodiment, the above-mentioned biological cells are cells colored/labeled with multiplex immunohistochemistry (MP-IHC), preferably fluorescent multiplex immunohistochemistry (fm-IHC), immunocytochemistry (ICC) or histological staining, preferably hematoxylin and eosin (H&E) staining.

In one preferred embodiment, the above-mentioned biological cells are immune cells, tumor cells, stem cells, blood cells (such as red blood cells (erythrocytes), white blood cells (leukocytes), and/or blood platelets (thrombocytes)), nerve cells (neurons), glia cells, muscle cells (myocytes), cartilage cells (chondrocytes), bone cells, skin cells, epithelial cells, fat cells (adipocytes), and/or germ cells (gametes).

In one more preferred embodiment, the above-mentioned biological cells are immune and/or tumor cells. The tumor cells are particularly cells expressing HER2, Programmed death ligand 1 (PD-L1), and/or Programmed death protein 1 (PD-1). The immune cells are particularly cells expressing CD3, CD4, CD8, CD45, CD45RO, and/or forkhead box P3 (FoxP3). However, also other cells may be analyzed.

According to at least one embodiment, the biological sample is a tumor sample such as a surgically removed primary tumor sample or a cell culture sample. The tumor sample may be a tumor sample section such as a formalin fixation and paraffin embedded tumor sample section.

The method for processing an image is further applicable to all (biological) tissue derived micrographs, including conventional immunohistochemistry (IHC) images, multiplex IHC (MP-IHC) images, conventional histological staining such as hematoxylin and eosin (H&E) staining, and other methods of tissue visualization. Specific use cases are the prediction of early relapse risk after cancer surgery and other diseases or disorders, disease staging in cancer and other diseases or disorders. Expected efficacy of treatment options e.g. after primary cancer surgery, or for refractory carcinoma. Expected survival time after cancer surgery etc.

Next, the grid data set is specified. The grid data set may be produced with the method for processing an image described herein. Therefore, all features disclosed in connection with the method for processing an image are also disclosed for the grid data set and vice versa.

According to at least one embodiment, the grid data set is indicative for the cell positions of all grid cells in an n-dimensional grid and also indicative for object features of objects of an initial image assigned to at least some of the grid cells. For example, an output image can be derived or is derived from the grid data set representing one or more object features at cell positions of grid cells to which the objects or object features are assigned. Some grid cells may not be assigned objects of the initial image. Those grid cells may be empty. Preferably, each grid cell is assigned one or more object features of at most one object of the initial image. Preferably, an object or object features from one object are assigned to at most one grid cell. Therefore, in the output image, each pixel or grid cell, respectively, represents exactly one or no object of the initial image and no two or more grid cells/pixels represent the same object.

According to at least one embodiment, the grid data set comprises an indicator. The indicator may be indicative for the origin of the initial image. E.g., the indicator is indicative for the patient or reference subject to which the initial image belongs. The indicator may also be indicative for the weight and/or the age and/or the sex of the patient/reference subject and/or the date or time at which the initial image was taken and/or the place at which the initial image was taken.

Next, the output image is specified. The output image may be produced with the method for processing an image specified herein. Therefore, all features disclosed in connection with the method for processing an image are also disclosed for the output image and vice versa. Moreover, the output image may be derived from a grid data set as specified herein. Therefore, all features disclosed for the grid data set are also disclosed for the output image and vice versa.

According to at least one embodiment, the output image is derived from a grid data set as specified herein.

Next, different uses of the grid data set and/or an output image are specified.

According to at least one embodiment, the grid data set and/or the output image are used for training or feeding a neural network. The grid data set and/or output image may be used as an input for the neural network. The neural network may be a neural network as specified above, i.e. the neural network as specified in connection with the method for processing an image.

According to at least one embodiment, the grid data set and/or the output image may be used for biomarker identification.

The grid data set and/or the output image of the present invention allow an easy analysis and interpretation. This helps in the discovery of new biomarkers for all applications.

Biomarkers are characteristic biological features that can be objectively measured and can indicate a normal biological or pathological process in the body. Biomarkers may be genes, gene products, specific molecules such as enzymes or hormones, spatial biomarkers, cell phenotypes like cell structures, cell morphologies, or other cell characteristics. Characteristic changes in biological structures are also used as medical biomarkers.

In particular, spatial biomarkers have been defined typically in the past using hand-crafted features, e.g. distance measurements between certain cell phenotypes, phenotype abundance in certain tissue areas, or other biomarkers focusing on the morphology on the tissue (vessel size or epithelium size). For example, the method for processing an image as described above aims at simplifying the interpretation of a trained neural network, such that someone does not need to study these images for weeks and months to come up with a hypothesis what a good spatial biomarker may be. Rather, the network learns the best discriminating feature from the data and the biomarker can be extracted from the trained network afterwards. The grid data set and/or the output image of the present invention may be obtained by this method and allow biomarker identification.

The grid data set and/or the output image of the present invention further offer advantages over other data and/or images. For example, they allow the determination of phenotype abundance: What cell phenotypes are present in what (relative) frequency? They further allow the determination of phenotype characteristics: What are the cell properties (marker values, size, shape)? They also allow the determination of the spatial distribution of phenotypes: Where are phenotypes located and what is their spatial relationship to each other (distances, co-localization, abundance in various tissue categories)? In addition, they allow the determination of large-scale morphology: What are the characteristics of the large-scale morphology of the tissue?

While the first three are in ascending order of complexity, the last one, morphology, is a special case since it does not necessarily depend on the individual cells.

For example, the method for processing an image as described above leads to a grid data set and/or output image which simplify biomarker types by reducing the problem to learning useful properties and relations of individual pixels, instead of large pixel areas in the image. Subsequently, interpretation of a trained model is simplified as well.

Usually, biomarkers can be classified into four types: diagnostic, prognostic, predictive, and therapeutic. Thus, the grid data set and/or the output image may be used for the identification of a diagnostic, prognostic, predictive, or therapeutic biomarker.

In this respect, the following should be noted. The term “diagnostic biomarker” may refer to a marker allowing the (early) detection of a disease or disorder, e.g. cancer, in a non-invasive way. The term “predictive biomarker” may refer to a marker allowing the prediction of the response of the patient to a targeted therapy and so defining subpopulations of patients that are likely going to benefit from a specific therapy. The term “prognostic biomarker” may refer to a marker having a clinical or biological characteristic that provides information on the likely course of the disease or disorder. It gives information about the outcome of the patient. The term “therapeutic biomarker” may refer to a marker which could be used as target for therapy.

Thus, the identification of a diagnostic, prognostic, predictive, or therapeutic biomarker allows to answer the following questions: Does the patient have a disease? (diagnostic biomarker) What is the likely course of the disease if the patient remains untreated? (prognostic biomarker) How will the patient response to a specific treatment? (predictive biomarker) What treatment should be given? What specific protein can be targeted during therapy? (therapeutic biomarker).

For example, immunotherapies have proven remarkably effective for treating cancer in some patients, but there remains a paucity of accurate biomarkers that can differentiate responders from non-responders. Identifying the patients most likely to respond to these therapies is an important step in ensuring optimal outcomes for all patients. To date, several assays have been developed with the potential to predict response based on genetic signatures, gene expression profiles, and immunohistochemistry. Although these assays are helpful in limited situations, there is a need for options that are better at predicting response across a larger percentage of cases.

Spatially resolved multiplex immunofluorescence, a type of biomarker assay, allows scientists to simultaneously analyse the expression of many proteins in individual cells within the tumor microenvironment, preserving critical information about which cells are influencing treatment response and how they are spatially distributed relative to each other.

The present inventors obtained/produced, in particular with the method for processing an image, a grid data set and/or an output image from biological cells coloured/labelled with multiplex immunofluorescence. Due to this technique the performance metrics of multiplex immunofluorescence could be improved.

According to at least one embodiment, the grid data set and/or an output image may be used for 3D tissue modelling.

Currently, 3D tissue visualization can be achieved using two methods: (1) using precision-cut tissue slices or (2) creation of z-stacking from consecutive 2D tissue sections. In (1), the tissue is cut into a comparatively thick slice on the order of a few hundred μm. After staining, this section can be visualized in 3D using confocal microscopy in which the focus of the microscope is used to move through the z direction. The obvious advantage of this is that the original 3D tissue stays intact. While z-resolution is typically smaller than the resolution in the xy plane, the resulting 3D models are “self-coherent”. In contrast, (2) uses consecutive tissue sections that are comparatively thin (˜3 μm). These sections are scanned individually and later aligned virtually using image registration techniques. Again, the z-resolution is limited, in this case by the section thickness. Furthermore, (non-rigid) image registration of the 2D sections may be difficult since the fragile thin samples can be warped and potentially show cracks at the sample edge. The final 3D model may not be “self-coherent”, depending on the quality of image registration.

Another problem with (2) is that naively, the 3D model is just a z-stack of “infinitely thin” 2D planes. To overcome this, the space between sections can be interpolated to create a dense model. Compared to (1) the models lack of self-coherency makes it difficult to visualize relevant 3D-aspects of the model, like vessels, epithelium etc. Therefore, this dense volume is best visualized by cutting through at arbitrary angles. In any case, visualization of (2) is troublesome.

The present inventors attempt at solving this visualization problem for (2) by using the method for processing an image for each 2D layer using the layer thickness as the target grid spacing. By using several consecutive tissue sections (e.g. cut at 3 μm thickness) and subsequent staining and scanning of all slides, image registration can be performed to align all scans and perform data compression in each scan individually to ultimately reconstruct a 3D model of the original tissue. This creates a 3D model as described above, e.g. of cubes of length of 3 μm in all 3 directions.

According to at least one embodiment, the grid data set and/or the output image are used for generating or modeling a synthetic grid data set and/or a synthetic output image. The generation or modeling of the synthetic grid data set/the synthetic output image is preferably based on information extracted from the grid data set and/or the output image. E.g., the synthetic grid data set/the synthetic output image is modeled based on a morphological structure extracted from the output image. The information extracted from the grid data set and/or the output image may be:
1) The large-scale morphology, e.g. the large-scale morphology of tissue categories. For example, the large-scale shape of the epithelium, the stroma, the vessels, and their location in relation to each other.
2) The object abundance, e.g. the phenotype abundance, particular in each tissue region/category. E.g. what biological cells are to be found in the epithelium (tumor cells, some invading tumor cells etc.), what biological cells are at the border of the tissue regions etc.?
3) The object features of the objects, e.g. the properties of the phenotypes. For example, what are the typical marker distributions of T cells, tumor cells etc.? What are their sizes, shapes etc.?
4) The neighborhood of the objects. E.g. do some objects have a specific local neighborhood or a specific colocalization with other objects, e.g. within the tissue categories? E.g. biological cells that tend to interact with each other, like immune cells that are interacting with other biological cells (e.g. tumor cells), need to be in contact with those biological cells to exchange information or perform their tasks.

Once, this information is extracted from the grid data set/output image, one can generate synthetic grid data/synthetic output images. For example, one can generate them in a top-down fashion with the following method:
First, an algorithm is created and/or used to imitate the extracted large-scale morphology, e.g. of the tissue.
Then, different regions are filled with appropriate objects, e.g. phenotypes, based on the extracted object abundance. For example, objects, like biological cells, are placed in the correct abundance at random locations in the different regions/categories.
Then, the objects get assigned object features based on the extracted object features of the objects. E.g. T cells will tend to have a high CD3 marker value.
Then, the positions of the objects are corrected based the extracted neighborhood of the objects, e.g. by making some biological cells explicitly touch each other.

The method described above is rule-based, i.e. a human can explicitly define the rules for how these synthetic grid data/synthetic output images should be constructed. The method can be performed with a computer.

The synthetic grid data set/the synthetic output image may have the same structure as the grid data set/the output image. E.g., the synthetic grid data set/the synthetic output image may comprise the same number of grid cells and/or the same size of grid cells and/or the same lattice constant as the grid data set/the output image. Preferably, also in the synthetic data set/synthetic output image, each grid cell is(are) assigned at most one object or object features of at most one object, respectively, and no more than one grid cell represents the same object.

Next, the device is specified. The device may comprise means for carrying out the method for processing an image specified herein. Preferably the device comprises one or more processors. For example, the device is a microscope. The device may be used for reading the initial image. The device may be configured for generating the object data set out of the initial image and for producing a grid data set and/or an output image from this object data set.

Next, the computer program is specified. The computer program may comprise instruction which, when the program is executed by a computer, cause the computer to carry out the method for processing an image specified herein.

Next, the computer-readable medium is specified. The computer-readable medium may comprise instruction which, when executed by a computer, cause the computer to carry out method for processing an image as specified herein.

Next, the neural network is specified. The neural network may be the neural network specified above. Particularly, the neural network may be trained with grid data sets and/or output images and/or a synthetic grid data sets and/or synthetic output images as specified herein.

Next, a method for diagnosing a disease or disorder in a patient is specified.

In particular, this method comprises the step of: comparing a grid data set and/or an output image (3) of a biological sample of a patient as obtained/defined above with at least one reference grid data set and/or at least one reference output image (3) of a biological sample of a reference subject as obtained/defined above, wherein this comparison allows diagnosing a disease or disorder in the patient.

The term “diagnosing a disease or disorder” may mean determining whether a patient shows signs of or suffers from a disease or disorder.

Instead of the term “at least one” also the term “one or more” may be used. For example, the term “one or more” may refer to 2, 5, 10, 50, 100, 150, 200, 250, 300, 400, 500, 1000, 2000, or 5000 grid data sets and/or output images of reference subjects.

The grid data set and/or output image of a reference subject may also be designated as reference grid data set and/or reference output image. Additionally or alternatively, the grid data set and/or output image may be used/analyzed/evaluated together with the reference grid data set and/or reference output image in order to diagnose a disease or disorder in the patient.

The reference grid data set and/or reference output image may be obtained like the grid data set and/or output image with the method for processing an image.

The reference subject is preferably a subject known to be healthy.

According to one embodiment, the diagnosis comprises
(a) determining the occurrence/presence of the disease or disorder,
(b) staging the disease or disorder,
(c) grading the disease or disorder, and/or
(d) segmentation of a patient suffering from the disease or disorder.

Staging and grading is often used in cancer. While the stage of a cancer describes the size of a tumor and how far it has spread from where it originated, the grade describes the appearance of the cancerous cells. Different types of staging systems are used for different types of cancer. Below is an example of one common method of staging:
Stage 0—indicates that the cancer is where it started (in situ) and has not spread.
Stage I—the cancer is small and has not spread anywhere else.
Stage II—the cancer has grown, but has not spread.
Stage III—the cancer is larger and may have spread to the surrounding tissues and/or the lymph nodes (part of the lymphatic system).
Stage IV—the cancer has spread from where it started to at least one other body organ; also known as “secondary” or “metastatic” cancer.

The grade of a cancer depends on what the cells look like under a microscope.

In general, a lower grade indicates a slower-growing cancer and a higher grade indicates a faster-growing one. The grading system that's usually used is as follows:
Grade I—cancer cells that resemble normal cells and aren't growing rapidly.
Grade II—cancer cells that don't look like normal cells and are growing faster than normal cells.
Grade III—cancer cells that look abnormal and may grow or spread more aggressively.

Segmentation means the classification of patients into certain patient groups on the basis of symptoms and disease or disorder characteristics.

Next, a method for prognosing the course of a disease or disorder in a patient is specified.

In particular, this method comprises the step of: comparing a grid data set and/or an output image (3) of a biological sample of a patient suffering from a disease as obtained/defined above with at least one grid data set and/or at least one output image (3) of a biological sample of a reference subject as obtained/defined above, wherein this comparison allows prognosing the course of the disease or disorder in the patient.

The term “determining the course of a disease or disorder” may mean determining the development of the disease or disorder over time, e.g. whether the disease or disorder worsens in the patient, does not worsen/is stable in the patient, or improves in the patient over time.

Instead of the term “at least one” also the term “one or more” may be used. For example, the term “one or more” may refer to 2, 5, 10, 50, 100, 150, 200, 250, 300, 400, 500, 1000, 2000, or 5000 grid data set and/or reference images of reference subjects.

The grid data set and/or output image of a reference subject may also be designated as reference grid data set and/or reference output image. Additionally or alternatively, the grid data set and/or output image may be used/analyzed/evaluated together with the reference grid data set and/or reference output image in order to prognose the course of the disease or disorder in the patient.

The reference grid data set and/or reference output image may be obtained like the grid data set and/or output image with the method for processing an image.

The reference subject is preferably a subject known to suffer a disease or disorder. Alternatively, the reference subject may be healthy.

According to one embodiment, the prognosis comprises
(a) predicting/estimating the severity of the disease or disorder,
(b) predicting/estimating the worsening of the disease or disorder,
(c) predicting/estimating the improving of the disease or disorder,
(d) predicting/estimating the survival time, and/or
(e) predicting/estimating the course of the disease or disorder with and/or without therapeutic treatment.

The term “determining the severity of a disease or disorder” may mean determining the degree and/or prognosis of a disease or disorder, e.g. whether the patient has a severe form of a disease or disorder with a poor prognosis or a mild form of a disease or disorder with a good prognosis. The disease or disorder may get worse or improve over time. This may prolong or shorten the survival time.

The term “predicting/estimating the course of the disease or disorder with therapeutic treatment” may also include the determination of the efficacy of a treatment.

Next, a method for determining whether a patient suffering from a disease or disorder will respond to a therapeutic treatment of said disease or disorder is specified.

In particular, this method comprises the steps of: comparing a grid data set and/or an output image (3) of a biological sample of a patient suffering from the disease as obtained/defined above with a grid data set and/or an output image (3) of a biological sample of a reference subject as obtained/defined above, wherein this comparison allows determining whether the patient suffering from the disease will respond to the therapeutic treatment of said disease or disorder.

Instead of the term “at least one” also the term “one or more” may be used. For example, the term “one or more” may refer to 2, 5, 10, 50, 100, 150, 200, 250, 300, 400, 500, 1000, 2000, or 5000 grid data set and/or reference images of reference subjects.

The grid data set and/or output image of a reference subject may also be designated as reference grid data set and/or reference output image. Alternatively or additionally, the grid data set and/or output image may be used/analyzed/evaluated together with the reference grid data set and/or reference output image in order to determine whether the patient suffering from the disease will respond to the therapeutic treatment of said disease or disorder.

The reference grid data set and/or reference output image may be obtained like the grid data set and/or output image with the method of processing an image.

The reference subject is preferably a subject known to be a responder of said therapeutic treatment. Alternatively, the reference subject is a subject known to be a non-responder of said therapeutic treatment.

The term “treatment”, in particular “therapeutic treatment” may refer to any therapy which improves the health status and/or prolongs (increases) the lifespan of the patient suffering from a disease or disorder. Said therapy may eliminate the disease or disorder in the patient, arrest or slow the development of the disease or disorder in the patient, inhibit the development of the disease or disorder in the patient, decrease the severity of symptoms in the patient suffering from the disease or disorder, and/or decrease the recurrence in a patient who currently has or who previously has had a disease or disorder.

The therapeutic treatment may encompass any form of medication, therapeutic treatment, or therapeutic intervention depending on the disease. There is, for example, the special case of “refractory carcinoma” patients. These are patients with some sort of tumor for which many therapies have been tried unsuccessfully and the doctors are “out of therapy options”. For these patients, more or less any treatment option is possible. Thus, the therapeutic treatment may also cover the off-label therapy/use. The term “off-label therapy/use” may mean the administration of almost any drug that was originally created for something completely different but which is repurposed to treat a patient if there is an indication that it might work.

According to one embodiment, the therapeutic treatment encompasses the administration of a drug. Therapy forms may by selected from the group consisting of chemotherapy, immunotherapy, and immunomodulatory therapy. In particular, the chemotherapy, immunotherapy, and immunomodulatory therapy encompasses the administration of a drug.

Next, a method for predicting relapse of a disease or disorder in a patient is specified.

This method comprises the step of: comparing a grid data set and/or an output image (3) of a biological sample of a patient as obtained/defined above with a at least one grid data set and/or at least one output image (3) of a biological sample of a reference subject as obtained/defined above, wherein this comparison allows predicting relapse of the disease or disorder in the patient.

Instead of the term “at least one” also the term “one or more” may be used. For example, the term “one or more” may refer to 2, 5, 10, 50, 100, 150, 200, 250, 300, 400, 500, 1000, 2000, or 5000 grid data set and/or reference images of reference subjects.

The grid data set and/or output image of a reference subject may also be designated as reference grid data set and/or reference output image. Alternatively or additionally, the grid data set and/or output image may further be used/analyzed/evaluated together with the reference grid data set and/or reference output image in order to predict whether the patient will have a relapse of the disease.

The reference grid data set and/or reference output image may be obtained like the grid data set and/or output image with the method for processing an image.

The term “disease or disorder” may refer to an abnormal condition that affects the body of an individual. A disease or disorder is often construed as a medical condition associated with specific symptoms and signs. A disease or disorder may be caused by factors originally from an external source, such as infectious disease, or it may be caused by internal dysfunctions, such as autoimmune disease. In humans, the term “disease or disorder” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behavior, and atypical variations of structure and function, while in other contexts and for other purposes these may be considered distinguishable categories. Diseases or disorders usually affect individuals not only physically, but also emotionally, as contracting and living with many diseases can alter one's perspective on life, and one's personality.

According to one embodiment, the disease or disorder is selected from the group consisting of cancer, an autoimmune disease, and a neurodegenerative disease.

The term “cancer” may describe the physiological condition in an individual that is typically characterized by unregulated cell growth. Examples of cancers include, but are not limited to, carcinoma, lymphoma, blastoma, sarcoma, and leukemia. More particularly, examples of such cancers include bone cancer, blood cancer lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma. The term “cancer” also comprises cancer metastases.

Hereinafter, the method for processing an image, the grid data set, the output image, the different uses of a grid data set and an output image, the device, the computer program, the computer-readable medium, the neural network, the method for diagnosing a disease, the method for prognosing the course of a disease, the method for determining whether a patient suffering from a disease will respond to a therapeutic treatment and the method for predicting relapse of a disease described herein will be explained in more detail with reference to drawings on the basis of exemplary embodiments. Same reference signs indicate same elements in the individual figures. However, the size ratios involved are not necessarily to scale, individual elements may rather be illustrated with exaggerated size for a better understanding.

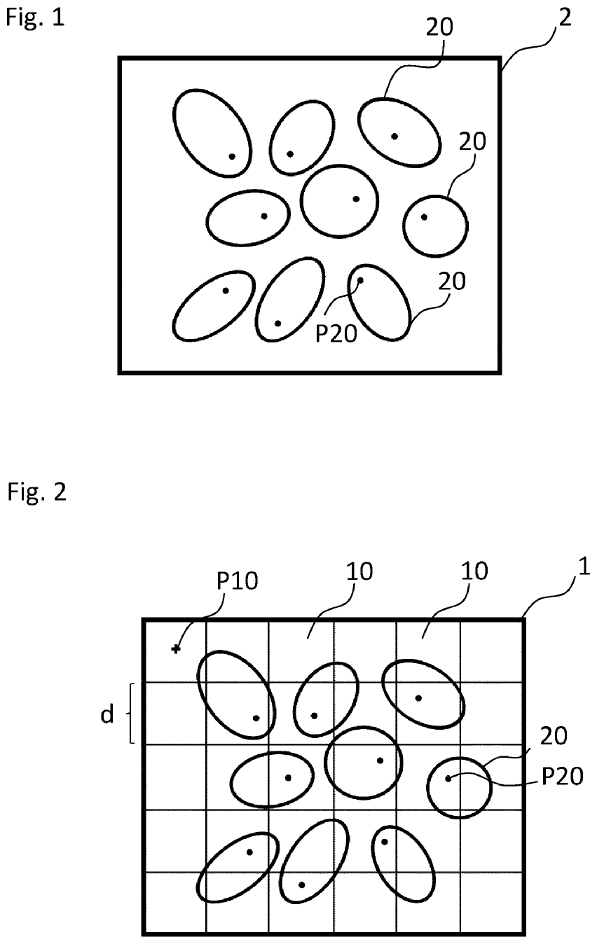

FIGS. 1 to 4 show different positions in a first exemplary embodiment of the method for processing an image,

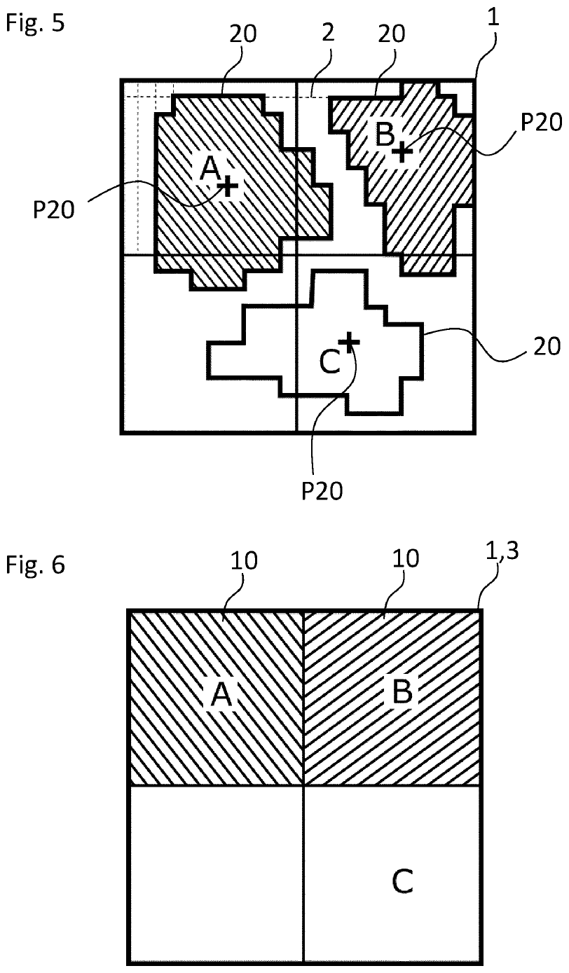

FIGS. 5 and 6 show different positions in a second exemplary embodiment of a method for processing an image,

FIGS. 7 to 10 show different positions in a third exemplary embodiment of the method for processing an image,

FIGS. 11 to 14 show different positions in a fourth exemplary embodiment of the method for processing an image,

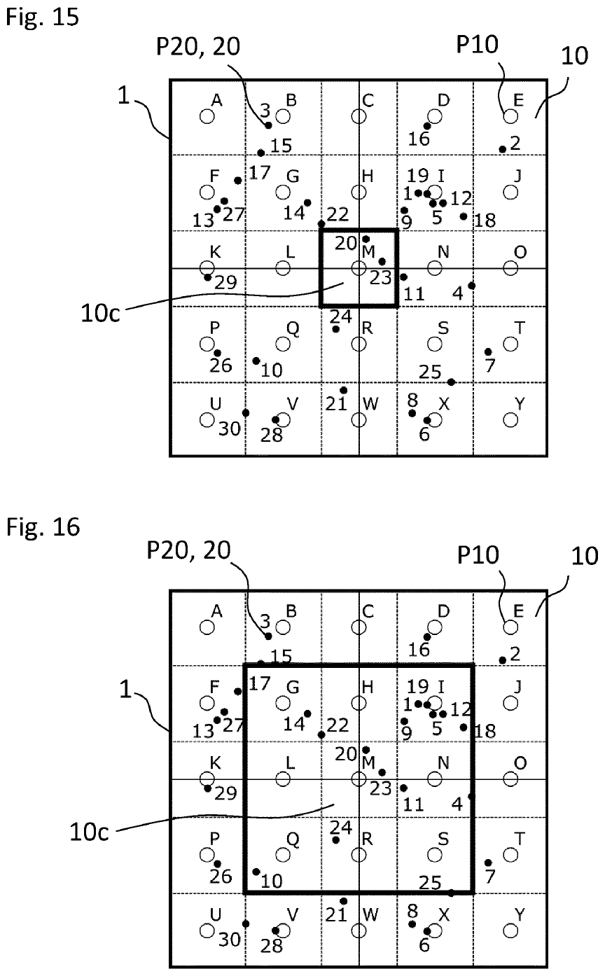

FIGS. 15 to 18 show different positions in a fifth exemplary embodiment of the method for processing an image,

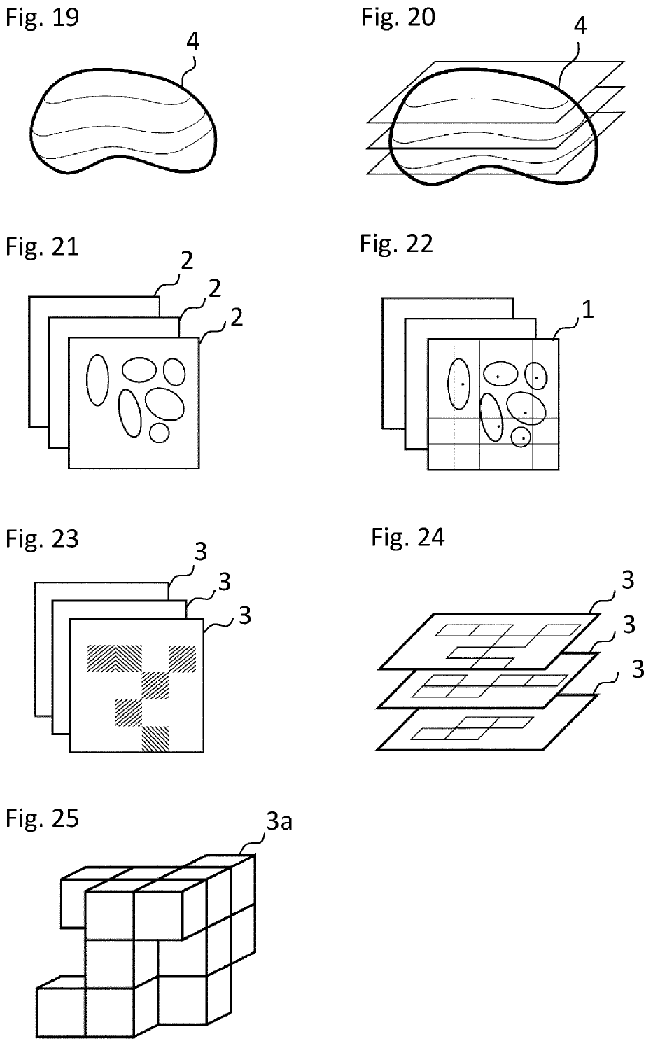

FIGS. 19 to 25 show different positions in sixth exemplary embodiment of the method for processing an image,

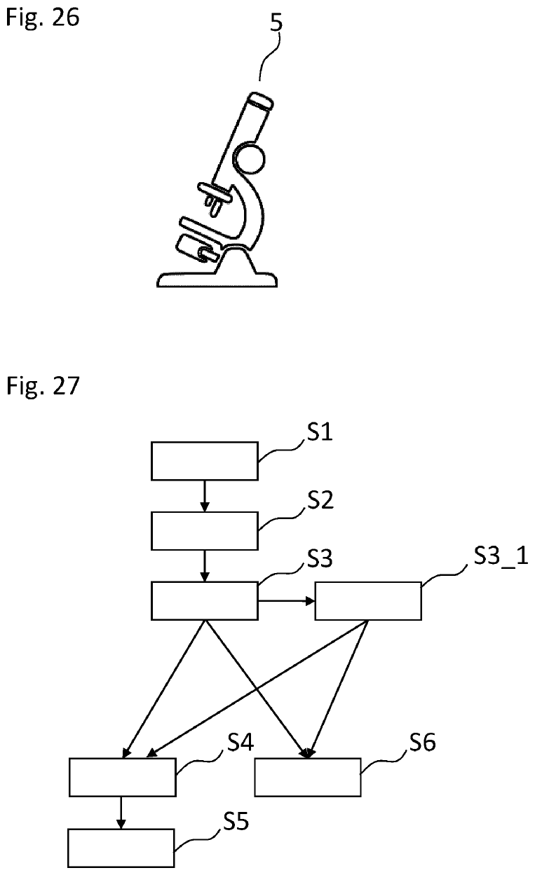

FIG. 26 shows an exemplary embodiment of a microscope,

FIG. 27 shows a flowchart of an exemplary embodiment of the method for processing an image.

FIG. 1 shows a first position in a first exemplary embodiment of the method for processing an image. An initial image 2 is provided. The initial image 2 shows a 2-dimensional spatial distribution of objects 20. The shapes of the objects 20 are visible. Furthermore, each of the objects 20 is assigned a position P20 on a one-to-one basis. For example, the positions P20 are the center-of-mass position of the respective objects 20.

The information about the 2-dimensional spatial distribution of the objects 20 may be stored in an object data set so that the 2-dimensional spatial distribution of the objects 20 may be derived from the object data set. The object data set may be obtained by identifying the objects 20 in the initial image 2, e.g. by using a commercial software. The objects 20 may be identified using the watershed algorithm. The positions P20 of the objects 20 may be stored in the object data set. Further information about the objects 20, e.g. the shape of the objects 20, or further features of the objects, herein called object features, may be stored in the object data set.

In this exemplary embodiment and in each other exemplary embodiment, the objects 20 may be biological cells and the initial image 2 may be an image of a biological tissue sample. For example, the initial image 2 is an immunohistochemistry image, particularly a multiplex immunohistochemistry image. The positions P20 may be the center of mass positions of the biological cells or the positions of the respective cell nucleus.

Alternatively, the initial image 2 may also be an image of one biological cell and the different objects 20 may be different regions or sections or parts of the biological cell. The different exemplary embodiments of the method may all be performed by a computer, i.e. may be a computer implemented.

FIG. 2 shows a second position in the method, in which a 2-dimensional grid 1 is assigned to the initial image 2 or the distribution of the objects 20, respectively. The grid 1 is assigned by virtually overlapping it to the initial image 2 or virtually aligning it with the initial image 2. The grid 1 comprises a plurality of grid cells 10 and each grid cell 10 is associated with a cell position P10, particularly on a one-to-one basis. The cell positions P10 may be, e.g., the center of mass positions of the cells 10. However, the cell positions P10 may also be the crossing points of the grid lines (grid nodes) or any other positions suitable for unambiguously indicating the positions of the grid cells 10. The grid cells 10 are represented by the meshes of the grid 1. The grid 1 is a regular grid with squared meshes or cells, respectively. However, also an irregular grid or a grid with differently shaped cells, e.g. rectangular or hexagonal grid cells 10, may be used.

The lattice constant d of the grid 1 may be chosen depending on the initial image 2 or the distribution of the objects 20 in the initial image, respectively. For example, the lattice constant d may be chosen depending on rho, wherein rho is the density of the objects 20 in the initial image 2. By way of example, the lattice constant d is chosen to be proportional to rho\hat{ }(−1/n), wherein n is the number of spatial dimensions of the initial image 2, which is 2 in the present case. Alternatively, the lattice constant d may be chosen depending on the expected fraction r of objects 20 which have to be deleted when assigning the objects 20 to grid cells 10, e.g. by using the formula introduced above. The fraction r may be set to at most 0.1%. In FIG. 2, the lattice constant is, e.g., 5 μm.

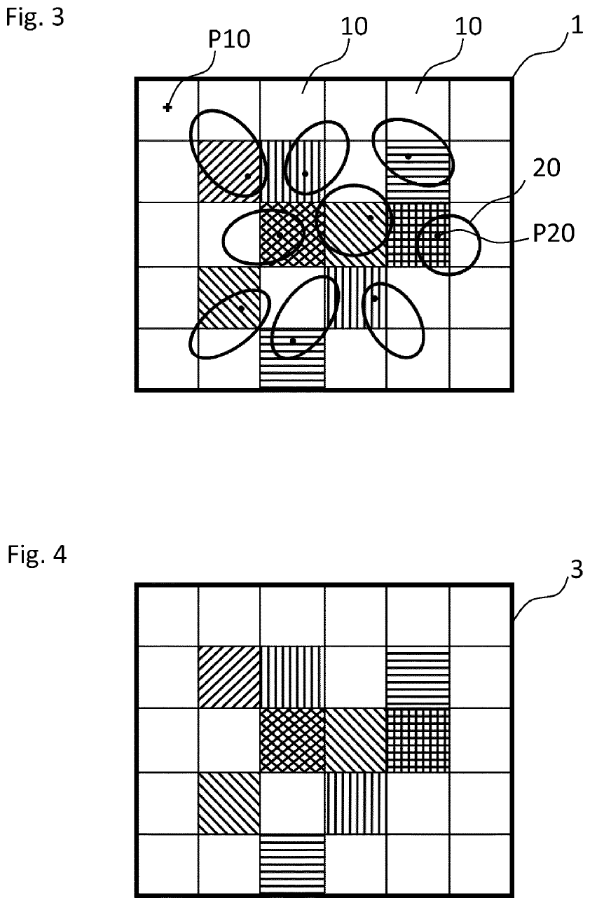

FIG. 3 shows a third position in the method, in which the objects 20 are assigned to the grid cells 10 depending on the positions P20 of the objects 20 relative to the cell positions P10. For example, an object 20 is assigned to that grid cell 10, for which the distance between the position P20 of the object 20 and the cell position P10 is the smallest. This assignment procedure (based on the smallest distance) is herein called first assignment procedure.

In FIG. 3, the assignment is indicated by the different hatchings. Particularly, the different objects 20 are not only characterized by the positions P20 but also by one or more object features. The object features may be one or more of: the size of the object 20, the shape of the object 20 or marker distribution properties in the object 20, e.g. average marker expression across the object area. These object features are represented by the different hatchings. In the shown case, the initial image 2 may be stained with six markers of interest (which is a multiplex image with six color channels). The marker intensity for color channel averaged over the entire biological cell may constitute one object feature. This may be done for all markers/color channels so that there are six object features. The different hatchings may represent all six object features.

The assignment of an object 20 to a grid cell 10 may be realized by assigning the one or more object features, e.g. all six object features, of this object 20 to the respective grid cell 10. In this way a grid data set is obtained in which the information about object feature(s) assigned to grid cells 10 is stored. For example, the information about the positions P20 of the objects 20 are not stored in the grid data set. Possible markers which may be used and cells which may be detected/labeled are described above.

FIG. 4 shows a fourth position in the method in which a 2-dimensional output image 3 is produced. The output image 3 may be obtained from the grid data set. The output image 3 is a visual representation of the grid data set. The output image 3 shows only the grid 1 and the object features (hatchings) of the objects 20 assigned to the respective grid cells 10. Some grid cells 10 were not assigned an object 20. These grid cells 10 remain empty. Therefore, the output image 3 is different from the initial image 2, e.g. in that the shape of the objects 20 is not shown anymore. Still the output image 3 is representative or indicative, respectively, for the initial image 2 in that it shows the object feature(s) of the objects 20 in grid cells 10, wherein the cell positions P10 are associated with the initial positions P20 of the objects 20.

FIG. 5 shows a position in a second exemplary embodiment of the method. Again, a 2-dimensional grid 1 has been spatially assigned to a 2-dimensional distribution of objects 20. The objects 20 are extractable from an initial image 2 having a first spatial resolution defined by a pixel size of the image 2. The pixel size is indicated by the dotted lines in FIG. 5. The pixel size of the initial image 2 may be at most 1 μm or at most 0.5 μm. The grid 1 has a second spatial resolution defined by the grid cell size or the lattice constant d. The grid cells 10, as in the previous pictures, are indicated by the solid lines. As can be seen, the pixel size of the initial image 2 is considerably smaller, e.g. by at least a factor of 5, than the grid cell size. In FIG. 5, the grid cell size may at least 5 μm.

FIG. 6 shows a further position in the second exemplary embodiment of the method after the objects 20 have been assigned to the grid cells 10 by assigning the object features (hatchings) to the respective grid cells 10. By assigning the objects 20 to the grid cells 10, the high-resolution of the initial image 2 is dispensed with and is converted into an output image 3 with a lower resolution. In particular, in the output image, each object 20 is represented by one single pixel, namely a grid cell 10, whereas in the initial image 2, each object 20 may be represented by a plurality of pixels. Nevertheless, relevant information, particularly object features and the rough position of the objects 20 is retained in the output image 3. Therefore, a data compression is achieved. In these compressed data, each object 20 is still uniquely identifiable, since every pixel (grid cell 10) corresponds to at most one object 20.

FIGS. 7 to 10 show a third exemplary embodiment of the method in which the assignment of the objects 20 to the grid cells 10 is explained in more detail.

FIG. 7 again shows a position in the method in which a grid 1 with grid cells 10 is spatially assigned to a distribution of objects 20. Each object 20 is assigned a position P20 and each grid cell 10 is assigned a cell position P10.

A first assignment procedure is executed in which objects 20 are assigned to the respective closest grid cell 10. For determining the closest grid cell 10, the Euclidean distance or the Manhattan distance between the position P20 of the object 20 and the cell position P10 may be calculated. As can be seen in FIG. 7, when using the first assignment procedure, two objects 20 would have to be assigned to the same grid cell 10. These objects 20 constitute conflicting objects 20c and the respective grid cell 10 constitutes a conflict grid cell 10c.

FIG. 8 shows a subsequent position of the method, in which a conflict resolution procedure is executed in order to assign the conflicting objects 20c to different grid cells 10. In this way, the objects 20 shall be assigned to grid cells 10 on a one-to-one basis. In the conflict resolution procedure, the conflicting object 20c which is closest to the conflict grid cell 10c is assigned to the conflict grid cell 10c and the other conflicting object 20c is assigned to the next closest, free grid cell 10. This approach is herein called priority shift.

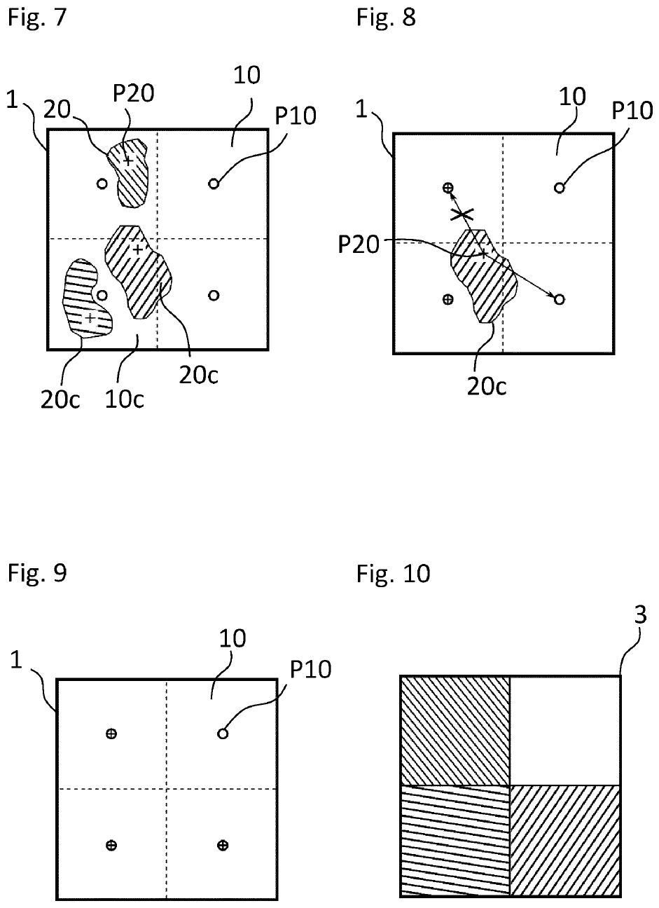

FIG. 9 shows the result after performing the conflict resolution procedure and FIG. 10 shows the output image 3 with the object features (different hatchings) of the respective objects 20 assigned to the grid cells 10.

FIGS. 11 to 14 show a fourth exemplary embodiment of the method in which the assignment of the objects 20 to grid cells 10 is explained in more detail.

In FIG. 11, a grid 1 with grid cells 10 is spatially assigned to a distribution of objects 20. The first assignment procedure is executed in which the objects 20 are assigned to the respective closest grid cell 10. As can be seen in FIG. 11, there are again two conflicting objects 20c which would have to be assigned to the same conflict grid cell 10c.

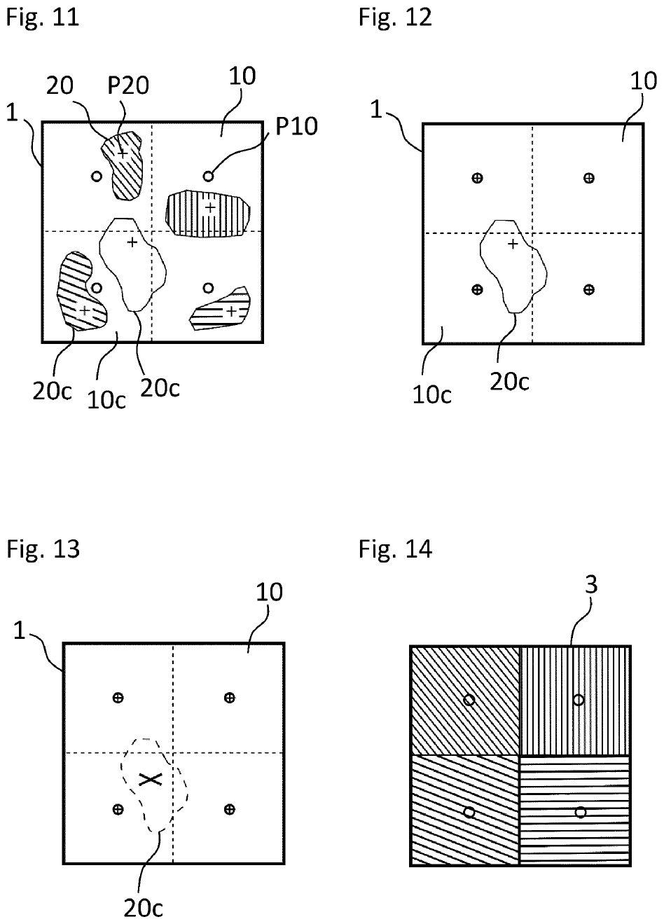

FIG. 12 shows a subsequent position after a conflict resolution procedure has been executed. In this conflict resolution procedure, the conflicting object 20c closest to the conflict grid cell 10c has been assigned to the conflict grid cell 10c (priority shift). Furthermore, in this conflict resolution procedure, it has been decided to not assign the other conflicting object 20c to any grid cell 10 since no other grid cell 10 in the neighborhood of the conflict grid cell 10c is free.

FIG. 13 shows a subsequent position, in which this remaining conflicting object 20c is deleted.

FIG. 14 shows the output image 3 with the object features (different hatchings) of the respective objects 20 assigned to the grid cells 10. The deletion of one object 20 has reduced the amount of information in the output image 3 compared to the initial image 2. This is the price which may have to be paid for the compression of the data.

FIGS. 15 to 18 show a fifth exemplary embodiment of the method in which the assignment of the objects 20 to the grid cells 10 is explained in more detail.

In FIG. 15, a grid 1 with grid cells 10 is spatially assigned to a distribution of objects 20. In order to increase the clarity of the picture, the shapes of the objects 20 are not indicated but only the positions P20 of the objects (see solid dots) are shown. The cell positions P10 of the grid cells 10 are indicated by circles. The different grid cells 10 are marked with letters A to Y, the different objects 20 are marked with numbers 1 to 30. Thus, there are more objects 20 than available grid cells 10.

In a first assignment procedure, it is again tried to assign each object 20 to the respective closest grid cell 10. However, this results in a conflict. For example, the objects 20 with the numbers 20 and 23 constitute conflicting objects 20c which would have to be assigned to the same grid cell 10 (with letter M) which therefore constitutes a conflict grid cell 10c.

In order to resolve this conflict, a conflict resolution procedure is executed. This conflict resolution procedure comprises the execution of the Hungarian algorithm in order to assign the conflicting objects 20c to grid cells 10 of a cell-set comprising the conflict grid cell 10c and one or more selected grid cells 10 in the neighborhood of the conflict grid cell 10c.

In a first approach, shown in FIG. 16, eight grid cells 10 in the neighborhood of the conflict grid cell 10c are selected to supplement the cell-set. The cell-set is a 3×3 subset of all grid cells 10 with the conflict grid cell 10c in the center (see solid square in FIG. 16). In a next step, an object-set is determined. The object-set comprises those objects 20 which would have to be assigned to the grid cells 10 of the cell-set when using the first assignment procedure. In the example of FIG. 16, the object-set comprises all objects 20 within the solid square. As can be seen in FIG. 16, the number k of objects 20 in the object-set is larger than the number m of grid cells 10 in the cell-set.

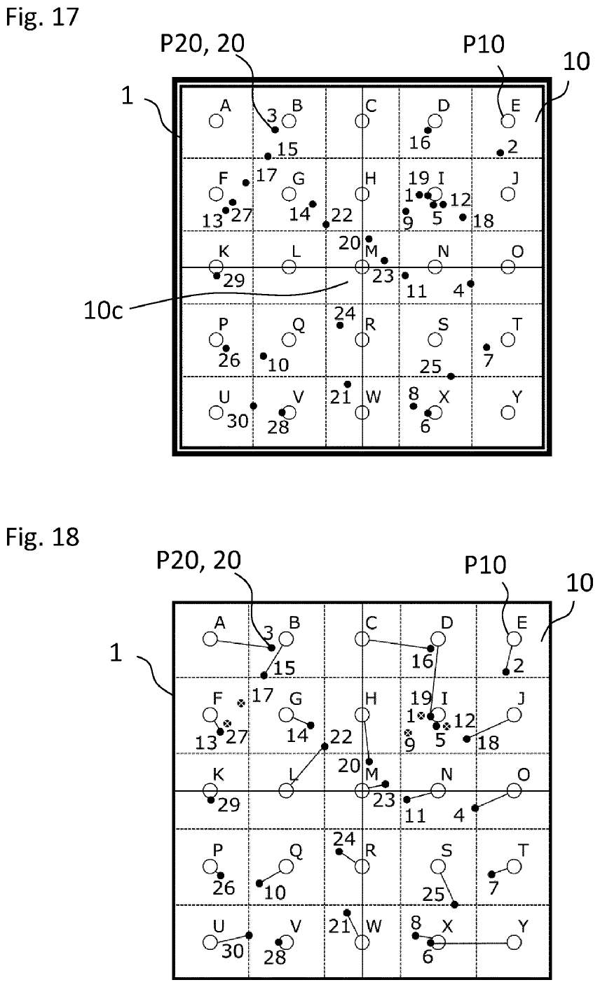

In the next step, shown in FIG. 17, the neighborhood of the conflict grid cell 10c is increased in order to increase the amount of selected grid cells 10 in the cell-set. In FIG. 17, the cell-set is now a 5×5 subset of all grid cells 10 with the conflict grid cell 10c in the center (see solid square). Next, again an object-set is determined comprising all objects 20 which would have to be assigned to the grid cells 10 of the new cell-set when using the first assignment procedure. In FIG. 17, it becomes apparent that the number k of objects 20 in the object-set is still larger that the number m of grid cells 10 in the cell-set.

The conflict resolution procedure may now be continued by further increasing the neighborhood of the conflict grid cell 10c in order to increase the number of selected grid cells 10 in the cell-set until the number k of objects 20 in the associated object-set is smaller or equal to the number m of grid cells 10 in the cell-set or until m reaches a predetermined maximum value m_max. In this exemplary embodiment, m_max is predetermined to 25 so that FIG. 17 shows already the maximum size of the cell-set.

The Hungarian algorithm is now executed for this cell-set and the objects of the associated object-set. If the number k of objects 20 in the object-set would be equal to the number m of grid cells 10 in the cell-set, the Hungarian algorithm could be solved as usual. However, in the present case, the number k of objects 20 in the object-set is larger than the number m of cells in the cell-set, namely by 5. In order to execute the Hungarian algorithm, virtual grid cells are included. In this way, the assignment matrix C used for the Hungarian algorithm is made square and comprises elements cij. The values of the elements cij depend on the distance dij between the i-th object 20 and the j-th grid cell 10. For example, the value of the elements cij is chosen to be proportional to dij\hat{ }x, wherein x is greater or equal 2. For the distance between an object 20 and a virtual grid cell, the value of cij may be chosen to be 0. After having performed the Hungarian algorithm, the objects assigned to virtual grid cells may be deleted.

FIG. 18 indicates the result of the Hungarian algorithm, namely the obtained assignment of the objects 20 to the grid cells 10. The lines indicate the assignment between the objects 20 and the grid cells 10. Some of the objects 20 have to be deleted, which is indicated by the crosses.

In case the number k of objects 20 in the object-set is smaller than the number m of grid cells 10 in the cell-set, virtual objects are included to make the assignment matrix C of the Hungarian algorithm squared. The grid cells 10 to which virtual objects are assigned when using the Hungarian algorithm may remain empty in the end.

FIGS. 19 to 25 shows a sixth exemplary embodiment of the method.

FIG. 19 shows a first position in the method, in which a sample 4, e.g. a biological tissue sample 4, is provided. In FIG. 20, the sample 4 is cut into slices. For example, each slice has a thickness between 1 μm and 20 μm, e.g. 5 μm.

FIG. 21 shows a position in which 2-dimensional initial images 2 of the slices are provided. For producing the initial images 2, a camera or microscope may be used. The different 2-dimensional initial images 2 may then be spatially aligned with each other so that they spatially fit to each other in order to form a correct 3-dimensional initial image. This may be done with commercial software.

FIG. 22 shows a position in which, for each initial image 2, a grid 1 is spatially assigned to the initial image 2. The lattice constant may be chosen to be equal to the thickness of the slices. Before that, objects 20, e.g. biological cells, may have been identified in the initial images 2 and for each initial image 2 a respective object data set may have been determined. The identification of the objects 20 in the initial images 2 may have been performed with a commercial software. For each initial image 2, a grid 1 with a different lattice constant may be used or the same lattice constant may be used for all initial images 2.

FIG. 23 shows a position in the method in which for each initial image 2 a respective grid data set and, accordingly, a respective 2-dimensional output image 3 has been produced, e.g. as described in connection with the previous exemplary embodiments.

FIGS. 24 and 25 show positions in which the output images 3 or the grid data sets obtained from the different initial images 2 are combined to produce a 3-dimensional output image 3a. For this purpose, the 2-dimensional grid cells 10 of each output image 3 are transformed into 3-dimensional grid cells. The 3-dimensional output images 3a is indicative for the initial images 2 and shows one or more object features at respective cell positions P10 in 3-dimensional space. Thus, the output image 3a is a compressed representation of the sample 4.

FIG. 26 shows a microscope 5. The microscope 5 may be configured to carry out the method according to any one of the previous exemplary embodiments. For example, the microscope 5 comprises one or more processors. A computer program may be stored in the microscope or the microscope may be connectable to a computer-readable medium, the computer program or computer-readable medium comprising instructions which, when executed by a computer, e.g. the microscope, cause the computer to carry out the method for processing an image.

FIG. 27 shows a flowchart of an exemplary embodiment of the method for processing an image. In a first step S1, an n-dimensional grid 1 is spatially assigned to a distribution of objects 20 in the n-dimensional space (see for example FIG. 2). In a second step, objects 20 are assigned to grid cells 10 depending on the spatial arrangement between the objects 20 and the grid cells 10 (see for example FIG. 3). In a step S3, a grid data set is produced by assigning one or more object features of the objects 20 to the respectively assigned grid cells 10 so that the grid data set is indicative for object feature(s) at cell positions P10 (see for example FIG. 4). An n-dimensional output image 3 is derived from the grid data set (step S3_1). The grid data set may comprise an indicator which is indicative for the origin of the initial image, e.g. a date on which the initial image was recorded or a name of a patient from which the initial image 2 was taken etc.

In step S4, the grid data set and/or the output image 3 is used for training an artificial neural network, e.g. a convolutional neural network. The neural network may be trained for producing an output, which may be one or more of: diagnosing a disease in a patient, prognosing the course of a disease in a patient, predicting whether patients suffering from a disease will respond to a therapeutic treatment of said disease, predicting relapse of a disease in a patient.

In a step S5, the trained neural network may be fed with a further grid data set and/or output image as an input in order to deliver an output based on this input. The further grid data set and/or the further output image may be obtained with the method steps S1 to S3/S3_1 but using a different initial image.

In an alternative step S6 the grid data set obtained in step S3 and/or the output image 3 obtained in step S3_1 may be used as an input for a neural network. The neural network may have been trained to produce an output based on the input, e.g. predicting relapse risk in cancer. The neural network may have been trained with one or more other grid data sets and/or other output images 3 and/or synthetic grid data sets and/or synthetic output images.

A diagnostic/prognostic/treatment monitoring method, e.g. colorectal cancer diagnosis/prognosis, which may be conducted on the basis of the information provided herein, in particular with respect to the image processing described herein, may be conducted as follows:

A patient with stage II colorectal cancer (CRC) gets surgery with curative intent to remove the primary tumor. Routinely, the tumor samples are analyzed (e.g. by a trained pathologist). Depending on the pathologists assessment and other parameters (how large was the tumor, how well differentiated, how many lymph nodes were removed etc.), the doctors decide upon further treatment, typically by following clear guidelines. One typical example, if the prediction is bad, is to administer adjuvant chemotherapy to further combat the tumor and to ensure that no part of the tumor remained in the body after surgery and/or that remaining tumor cells are not able to spread again, eventually leading to metastasis in other organs and typically worse patient outcome.

The reason why adjuvant chemotherapy after primary tumor surgery is not just given to every patient is that typically only 30% of stage II CRC patients even suffer from a relapse. This means, 70% of patients would get unnecessary chemotherapy, which of course has severe side effects. However, deciding which patient is at risk and would benefit from adjuvant chemotherapy is unclear, and the existing guidelines and assessments do not show high precision.

The information provided herein, in particular with respect to the image processing described herein, helps to determine which patient should get adjuvant chemotherapy and which is likely to experience a relapse.

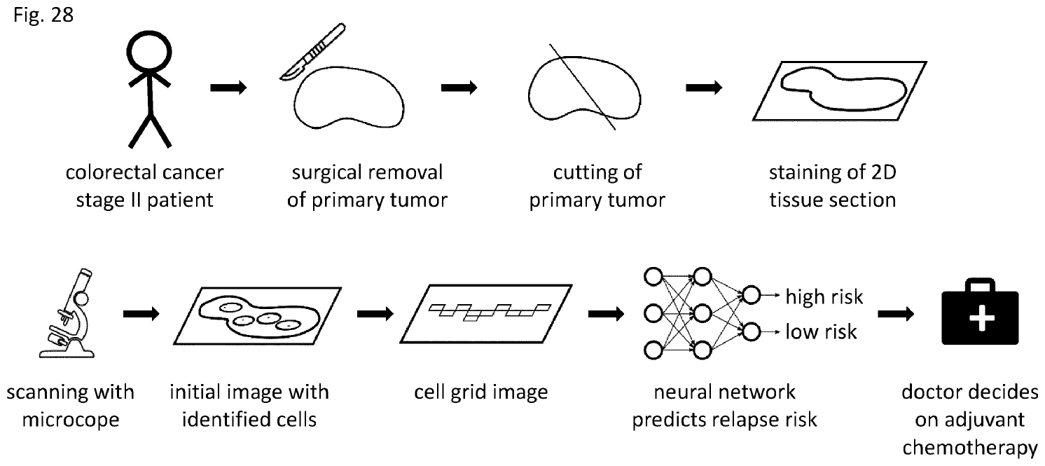

A schematic overview of this procedure is presented in FIG. 28.

The invention described herein is not limited by the description in conjunction with the exemplary embodiments. Rather, the invention comprises any new feature as well as any combination of features, particularly including any combination of features in the patent claims, even if said feature or said combination per se is not explicitly stated in the patent claims or exemplary embodiments.

### REFERENCE NUMERALS

1 grid
2 initial image
3 output image
3a output image
4 sample
5 microscope
10 grid cell
20 object
10c conflict grid cell
20c conflicting object
P10 cell position
P20 position of object 20
S1 . . . S6 method steps
d diameter of a grid cell 10

1. A method for processing an image comprising the steps:
spatially assigning an n-dimensional grid (1) to a distribution of objects (20) in the n-dimensional space, wherein
n is greater or equal 2,
the distribution of the objects (20) is derivable from an object data set and the object data set is indicative for an arrangement of the objects (20) in an n-dimensional initial image (2),
the grid (1) comprises grid cells (10) and each grid cell (10) is associated with a cell position (P10) in the grid (10),
assigning objects (20) to grid cells (10) depending on the relative spatial arrangement between the objects (20) and the grid cells (10).

2. The method according to claim 1, wherein
the object data set is indicative for the positions (P20) of the objects (20) in the n-dimensional initial image (2),
the objects (20) are assigned to the grid cells (10) depending on the positions (P20) of the objects (20) relative to the cell positions (P10).

3. The method according to claim 2, wherein
objects (20) are assigned to grid cells (10) on a one-to-one basis.

4. The method according to claim 1, wherein, for assigning the objects (20) to grid cells (10),
a first assignment procedure is executed in which objects (20) are assigned to the respective closest grid cell (10) and,
if, in the first assignment procedure, two or more objects (20) are to be assigned to the same grid cell (10), these objects (20) constitute conflicting objects (20c), the respective grid cell (10) constitutes a conflict grid cell (10c) and a conflict resolution procedure is executed in order to assign the conflicting objects (20c) to different grid cells (10) and/or to decide to not assign at least one of the conflicting objects (20c) to any grid cell (10).

5. The method according to claim 1, wherein
at least some objects (20) are assigned to grid cells (10) using the Hungarian algorithm,
an assignment matrix C is used with the values of the elements cij of the matrix C depending on the distance between the i-th object (20) to the j-th grid cell (10).

6. The method according to claim 5, wherein
the values of the elements cij are proportional to the squared distance between the i-th object (20) to j-the grid cell (10),
the distance between the i-th object (20) to j-th grid cell (10) is the Euclidean distance or the Manhattan distance.

7. The method according to claim 4, wherein
the conflict resolution procedure comprises the execution of the Hungarian algorithm in order to assign the conflicting objects (20c) to a cell-set comprising the conflict grid cell (10c) and one or more selected grid cells (10) in the neighborhood of the conflict grid cell (10c).

8. The method according to claim 7, wherein, when executing the conflict resolution process,
an object-set is determined comprising objects (20) which would have to be assigned to the grid cells (10) of the cell-set when executing the first assignment procedure,
afterwards, only the objects (20) of the object-set are assigned to the grid cells (10) of the cell-set by using the Hungarian algorithm.

9. The method according to claim 8, wherein
if the number k of objects (20) in the object-set is larger than the number m of grid cells (10) in the cell-set, the neighborhood of the conflict grid cell (10c) is increased in order to increase the amount of selected grid cells (10) until m is greater or equal k or until m reaches a predetermined maximum value m_max.

10. The method according to claim 9, wherein
m_max is smaller than the total number of grid cells (10) in the grid (1),
m_max is at most 100 or at most 49.

11-13. (canceled)

14. The method according to claim 1, wherein
the object data set is indicative for one or more object features being characteristic for the objects (20).

15. The method according to claim 14, further comprising
producing a grid data set by assigning one or more object features of the objects (20) to the grid cells (10) to which the objects (20) are assigned,
wherein the grid data set is indicative for the cell positions (P10) of all grid cells (10) of the grid (1) and indicative for which object features are assigned to which grid cell (10).

16. The method according to claim 15, further comprising
producing an n-dimensional output image (3) depending on the grid data set, wherein the output image (3) is indicative for the initial image (2) and shows one or more object features at the respective cell positions (P10).

17-31. (canceled)

32. A grid data set produced with the method according to claim 15.

33. (canceled)

34. An output image (3) produced with the method according to claim 16.

35-39. (canceled)

40. A device comprising means for carrying out the method according to claim 1.

41. A computer program comprising instructions which, when the program is executed by a computer, cause the computer to carry out the method according to claim 1.

42. A computer-readable medium comprising instructions which, when executed by a computer, cause the computer to carry out the method according to claim 1.

43. A neural network trained with the grid data set and/or the output image (3) according to claim 32.

44. A method for diagnosing a disease in a patient comprising the step of: comparing a grid data set and/or an output image (3) of a biological sample of a patient according to claim 32 with at least one reference grid data set and/or at least one reference output image (3) of a biological sample of a reference subject according to claim 32, wherein this comparison allows diagnosing a disease in the patient.

45-54. (canceled)
